(19) United States
(12) Patent Application Publication
(10) Pub. No.: US 2010/0318051 A1
(43) Pub. Date: Dec. 16, 2010

(54) Disposable menstrual Panties for little girls

(76) Inventors: Patricia Ann McCourt, Del Rio, TX (US); Diana Maria Jameson, Del Rio, TX (US)

Correspondence Address: Patricia McCourt, 515 Virginia St., Del Rio, TX, 78840, US

(21) Appl. No.: 12/802,648

(22) Filed: Jun. 11, 2010

Related U.S. Application Data
(60) Provisional application No. 61/187,074, filed on Jun. 15, 2009.

(51) Int. Cl. A61F 13/49; A61F 13/53

(52) U.S. Cl. 604/374; 604/385.101

(57) ABSTRACT

A disposable menstrual panty for little girls. An undergarment configured to be worn as a panty for absorption of menstrual fluid. The panty is made out of breathable non-porous material. The crotch area has a build in sanitary napkin made out of a mesh material used in sanitary napkins to help pull the moisture of fluids away from the young girls body, helping them to feel drier. The leg areas are made of flexable gathers that will cling around the legs for a secure fit (to prevent leakage). The sides of the light weight hip hugger panties will have Velcro sides so that they can be replaced or removed easily. The disposable menstrual panty for little girls is to be worn by a young female who is just beginning to experience her first years of menstruating. The undergarment with the build in sanitary napkin, while providing additional layers and absorbent materials will provide confidence, and longer wear for young females.

### BACKGROUND OR PROBLEM

Beginning a menstrual cycle can be an intimidating time in a young girls life. A female may not know the correct way to adhere a sanitary napkin to an undergarment. They may also not realize how often the item may need to be changed. Conventional sanitary napkins may be built for a more adult physique. Excess material of the napkin may rub against a young ones legs, creating discomfort and possibly rashes. Girls may find keeping a sanitary napkin in place throughout the day to be an arduous task. Slight shifts can result in stained underwear and embarrassment. These stains may be difficult to remove, often times forcing the female to discard the article. Purchasing replacement undergarments can be expensive. Having a disposable garment specifically made to fit the needs of a young, menstruating girl may assist them in feeling more comfortable when becoming acclimated with the process.

### SOLUTION

Disposable Menstrual Panties for Little Girls provide young females with a discreet and comfortable garment that can be worn in place of bulky, sanitary napkins that may be intended for use on an adult body. These unique products may assist a female in feeling secure and assured during menstruation by wearing an item specially designed to accurately fit her young body. These innovative items are ideal for use by any young female that may incur her period at a young age in which traditional hygiene products may be ill fitting or uncomfortable to don.

### DETAILED DESCRIPTION OF INVENTION

Disposable Menstrual Panties for Little Girls offer young females a personal hygiene product tailor made for their small statures. These products may be a hip-hugger style to snugly and discreetly fit under clothing. The item may be similar to a standard panty, but made from a breathable, absorbent material. It may have flexible elastic around the leg areas for a secure fit and to prevent leakage. The crotch area may be an absorbent material, similar to that found in a sanitary napkin. Velcro sides above leg openings for easy removal and replacement. These items may be produced in various styles, designs, patterns, colors and sizes to accommodate user preferences and needs. After use, the item may be disposed of in a trash receptacle. The exact dimensions, materials used for construction and method of operation of Disposable Menstrual Panties for Little Girls may vary upon manufacturing.

### DETAILED DESCRIPTION OF DRAWINGS ATTACHED

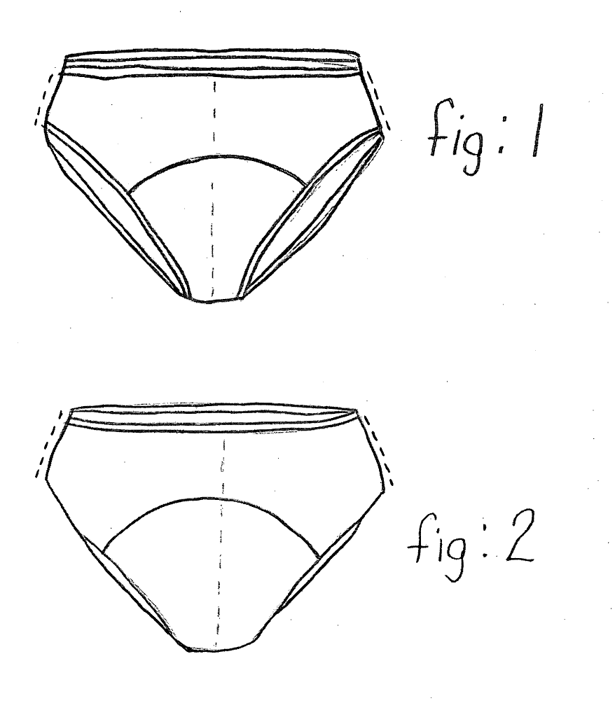

FIG. 1. Shows a front view of what panty looks like.

FIG. 2. Shows the back view of what panty looks like.

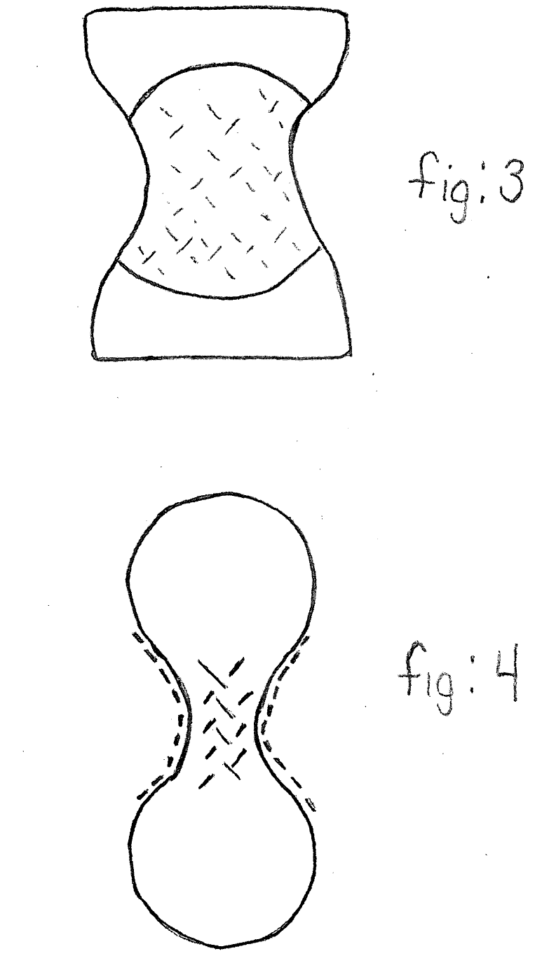

FIG. 3. Shows the inside view of the unit.

FIG. 4. Shows the sanitary napkin that would be built into the unit.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

Having thus described the aforementioned invention, we claim:

1. A disposable menstrual panty for little girls for absorption of menstrual fluid excreted by a wearer, comprising:
A front interior portion attached to a rear interior portion along opposed side portions of a panty said front and rear interior portions having an interior surface;
A crotch interior portion joining said front interior portion and said rear interior portion and said rear interior portion of said panty;
A waistband of generally continuous material;
Two leg openings through said opposed side portions and adjacent said crotch portion, each of said two leg openings having an interior border;
An exterior layer of said panty, said exterior layer of breathable non-porous material;
A plurality of layers disposed on interior surfaces of said panty including;
A middle layer of absorbent materials having a front layer, a crotch layer and rear layer positioned on each of respective interior surfaces of said crotch portion of said panty;
A crotch area has a build in sanitary napkin made out of a mesh material commonly used in sanitary napkins to help pull the moisture of fluids away from the young female body;
Strips of absorbent materials attachable along said interior border of each of said two leg openings, said at least one strip of absorbent materials being generally continuous along each interior border of each of said two leg openings;
Velcro sides above two leg openings to easily remove panty when ready to dispose of;

2. The disposable menstrual panty for little girls of claim 1 wherein said undergarment including said exterior layer and said waistband being composed of cellulose-based material, said cellulose-based material having a plurality of stretchable filaments therein, said waistband is stretchable in a width dimension, said interior surface of said front interior portion, rear interior portion, and said crotch interior portion having a layer of absorbent materials thereon;

3. The disposable menstrual panty for little girls of claim 2 wherein said interior layer of absorbent materials and said at least one strip of absorbent materials along each interior border are composed of a cellulose-based material having at least one layer of generally continuous absorbent materials along interior surfaces of said front portion, said rear portion, said crotch portion, and each interior border of said panty;

4. The disposable menstrual panty for little girls would have a build-in sanitary napkin for extra protection of claim 1 made of a mesh material commonly used in sanitary napkins to pull moisture away from female body, hourglass shape sanitary napkin would expand to absorbent strips attached along two leg openings, half way up front and rear of absorbent panty

5. The disposable menstrual panty of claim 1 wherein said middle layer of absorbent materials is integrally attached to interior surface of said front portion, rear portion, and said crotch portion to provide a continuous interior layer of absorbent materials composed of an absorbent polymer layer;

6. The disposable menstrual panty of claim 1 wherein at least one strip of absorbent materials along each interior border of each of said leg openings in composed of a cellulose-bases absorbent layer or an absorbent polymer layer;

7. A disposable menstrual panty for absorption of menstrual fluids excreted by a wearer, comprising;
A panty having an exterior layer of breathable, non-porous and stretchable material, said panty including an interior surface of said exterior layer, said interior surface including a front portion attached to a rear portion, a crotch portion with a build in sanitary napkin, two leg openings on opposed sides of said crotch portion, velcro sides above leg openings for easy removal;
An interior circumferential border of absorbent materials positioned along interior surfaces of each of said two leg openings;
A middle layer of absorbent materials positioned on each of an interior surface of said front portion, rear portion, and said crotch portion said middle layer extended laterally to overlap said interior circumferential border of absorbent materials positioned along said interior faced surfaces of each of said two leg openings.

8. The disposable menstrual panty for little girls of claim 7 wherein said panty including said exterior layer and said waistband being composed of cellulose-based material, said cellulose-based material having a plurality of stretchable filaments therein, said waistband is stretchable in a width dimension, said interior surface of said front portion, said rear portion, and said crotch portion having a layer of absorbent materials thereon:
